(19) United States
(12) Patent Application Publication
(10) Pub. No.: US 2017/0340199 A1
(43) Pub. Date: Nov. 30, 2017

(54) PRESSURE AUGMENTED SCOPING PROCEDURE PADDLES

(71) Applicant: Robin Page, Houma, LA (US)

(72) Inventor: Robin Page, Houma, LA (US)

(21) Appl. No.: 15/169,142

(22) Filed: May 31, 2016

(51) Int. Cl. A61B 1/31; A61B 1/00

(52) U.S. Cl. CPC A61B 1/31; A61B 1/00131

(57) ABSTRACT

The instance pressure paddles decrease the physical stress on healthcare providers. It allows healthcare professionals to apply focused, even pressure on the human lower abdomen while rotating positions during a colonoscopy procedure. The device can reduce injury to both providers and patients. The device allows procedure times to become more predictable which in turn improves scheduling for patients and teams performing the procedure.

### FIELDS OF THE INVENTION

The disclosure as detailed herein is in the technical field of medical procedures. More specifically, the present disclosure relates to the technical field of hand held physical devices. Even more specifically, the present disclosure relates to the technical field of compression devices.

### DESCRIPTION OF RELATED ART

Health care professionals performing colonoscopies face the challenge of properly guiding the colonoscope through the length of the colon in order to properly observe one's colon for health risks. The current process for guiding a colonoscope requires one to physically manipulate the lower abdomen in order to prevent the colonoscope from looping or getting off track. A health care professional guiding a colonoscope must be able to apply broad focused pressure with their hands as well as shift positions during the process. The number of procedures performed can be limited by the stamina of a health care professional, leading to fatigue and possible injury for the person performing the procedure. Patients can face the risk of a colon perforation or other injuries if the procedure is not performed correctly. Colonoscopies are essential diagnostic procedures for disease prevention and/or evaluation of colon related issues or injuries. The human nature of guiding the colonoscope can lead to slower or unpredictable procedure times and scheduling.

### GENERAL SUMMARY OF THE INVENTION

The instance pressure paddles decrease the physical stress on healthcare providers. It allows healthcare professionals to apply focused, even pressure on the human lower abdomen while rotating positions during a colonoscopy procedure. The device can reduce injury to both providers and patients. The device allows procedure times to become more predictable which in turn improves scheduling for patients and teams performing the procedure.

An embodiment of the instant invention allows one to decrease physical stress on nurses during colonoscopy.

Yet another embodiment of the invention allows one to press through intervening tissue more easily by using fulcrum of handles for pressure.

Yet another embodiment of the invention allows one to rapidly shift positions during the procedure.

Yet another embodiment of the invention allows one to change pressure spots without changing surface area so that pressure breadth is more consistent.

Yet another embodiment of the invention allows one to have a focused enough curve in order to progress through intervening tissue more easily and apply pressure to colon.

Yet another embodiment of the invention allows one to utilize a lightweight device.

Yet another embodiment of the invention allows one to prevent medical staff injury.

Yet another embodiment of the invention allows one to improve staff social relations.

Yet another embodiment of the invention allows one to improve the speed of colonoscopies.

Yet another embodiment of the invention allows one to improve the number of colonoscopy completions.

Yet another embodiment of the invention allows one to improve the accuracy of patient diagnostics to have a more efficient and better colonoscopies resulting in better health.

Yet another embodiment of the invention allows one to decrease colon injury and the liability associated with outpatient procedures.

Yet another embodiment of the invention allows one to have better gi-related diagnosis and treatment for patients with colon pathologies.

Yet another embodiment of the invention allows one to have a disposable or easily cleanable device.

An additional embodiment allows one to decrease the wear and tear on the colonoscope.

### DESCRIPTION OF FIGURES

FIG. 1 is a diagram view which shows the overall method of use for the device.

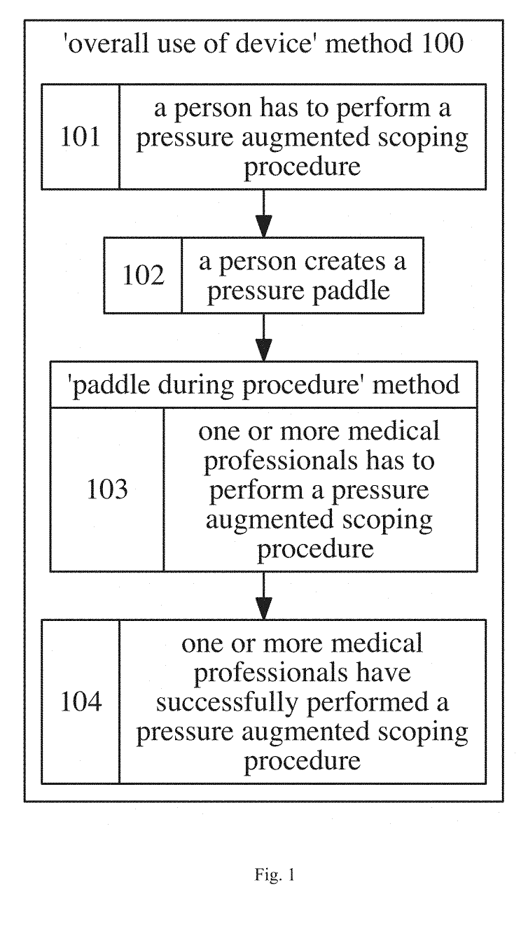

FIG. 2 is a diagram view which shows how to use the paddle during the procedure.

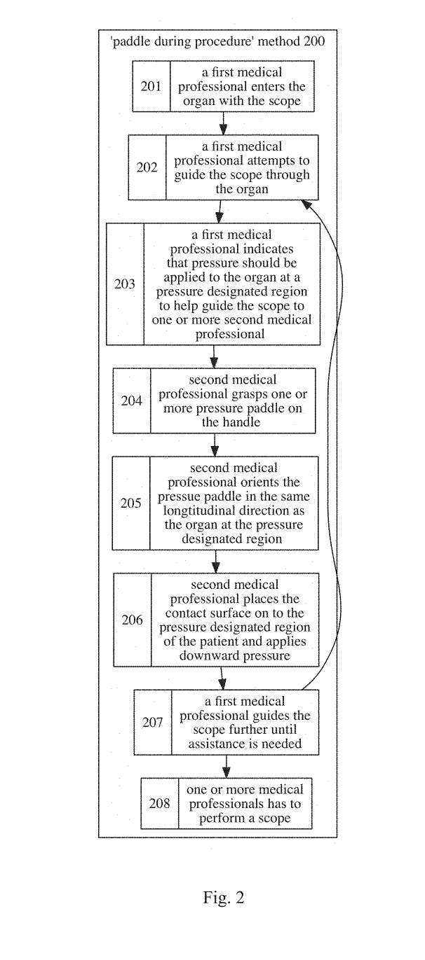

FIG. 3 is a perspective view which shows the device being used on a patient.

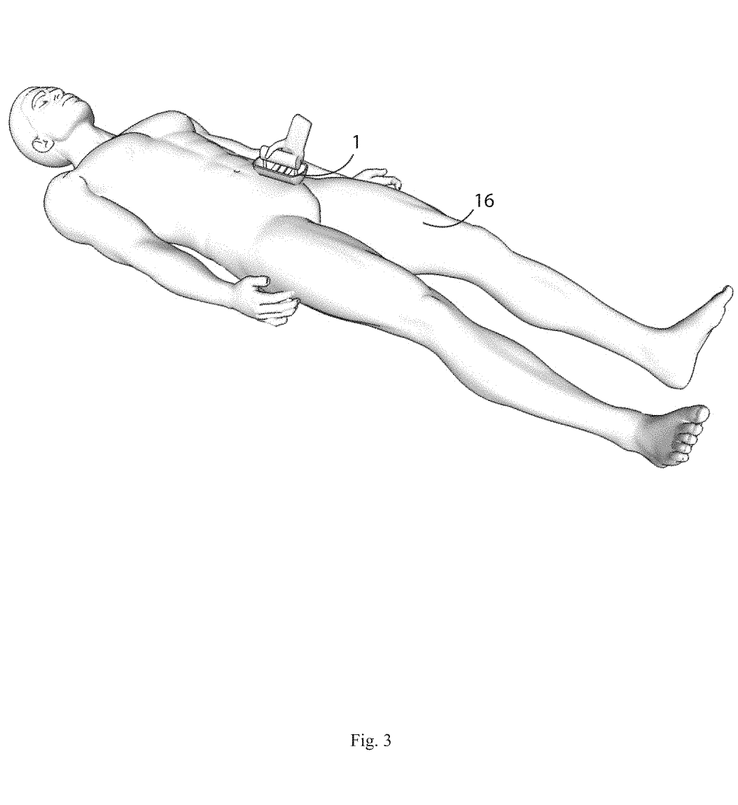

FIG. 4 is a perspective view which shows the device being used on a patient.

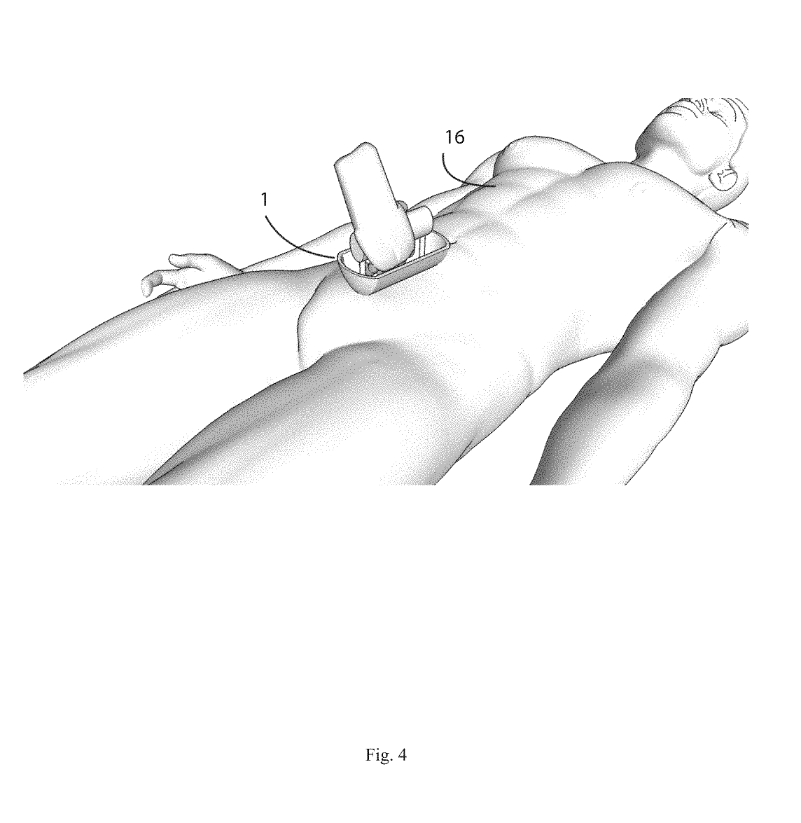

FIG. 5 is a perspective view which shows the device being grasped by a medical professional.

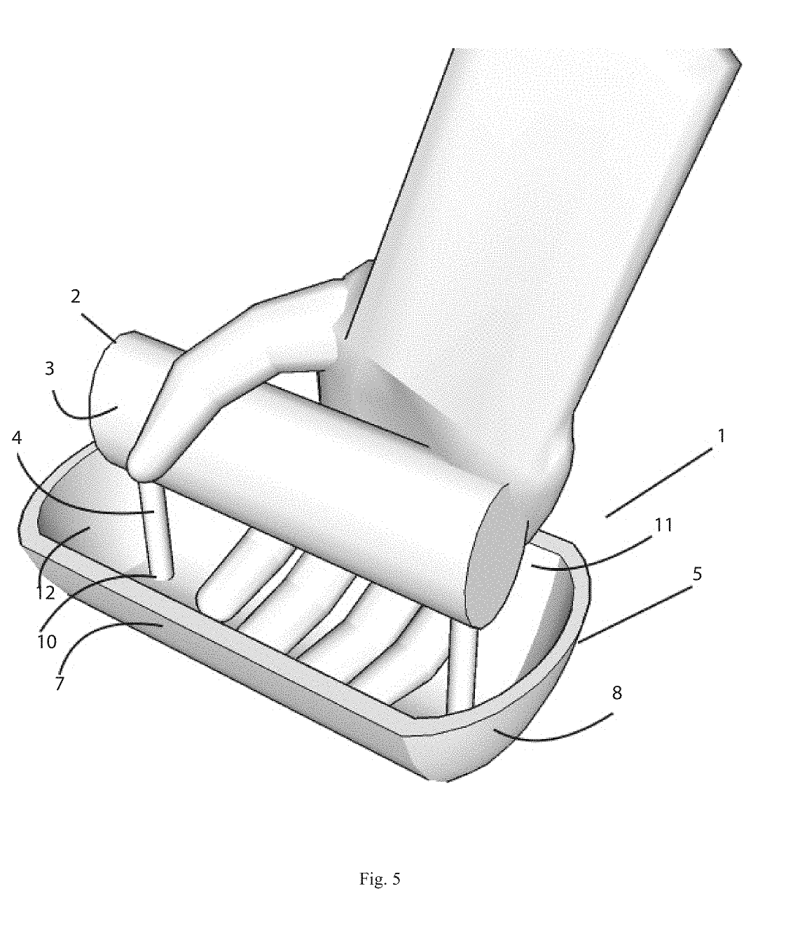

FIG. 6 is a perspective view which shows the pressure block size in relation to the handle size.

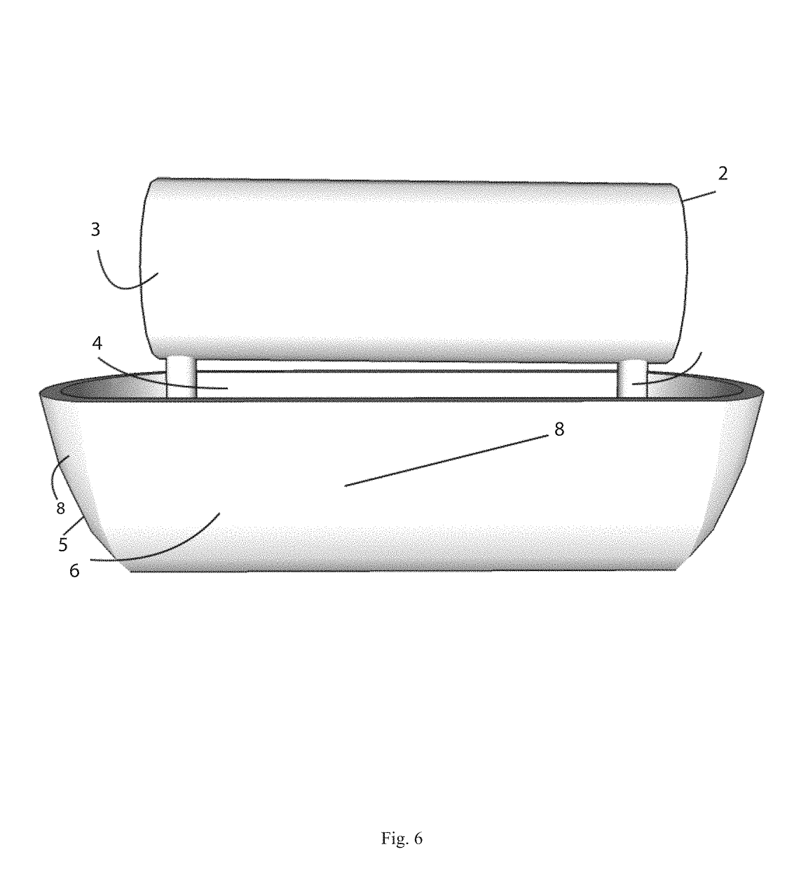

FIG. 7 is a perspective view which shows the contact surface of the pressure block.

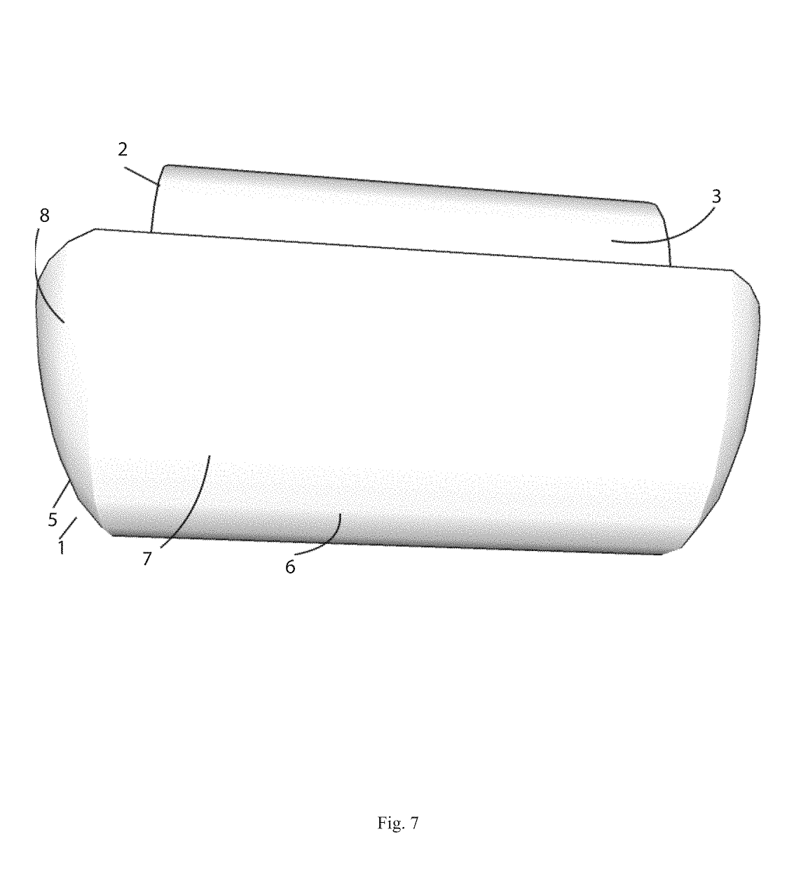

FIG. 8 is a perspective view which shows the distal ends of the pressure paddle.

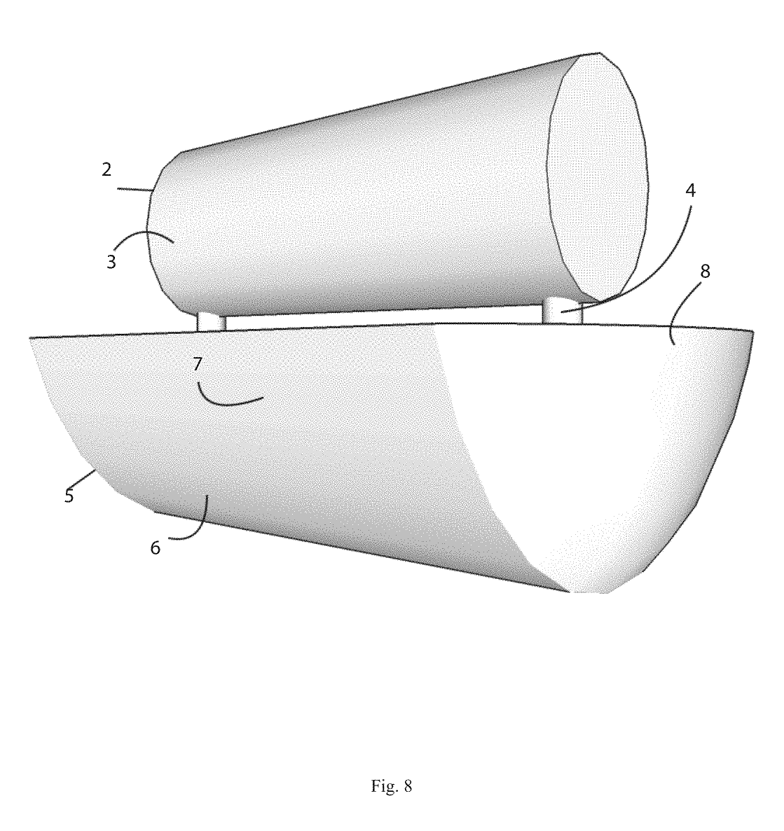

FIG. 9 is a perspective view which shows the superior surface and connector arms.

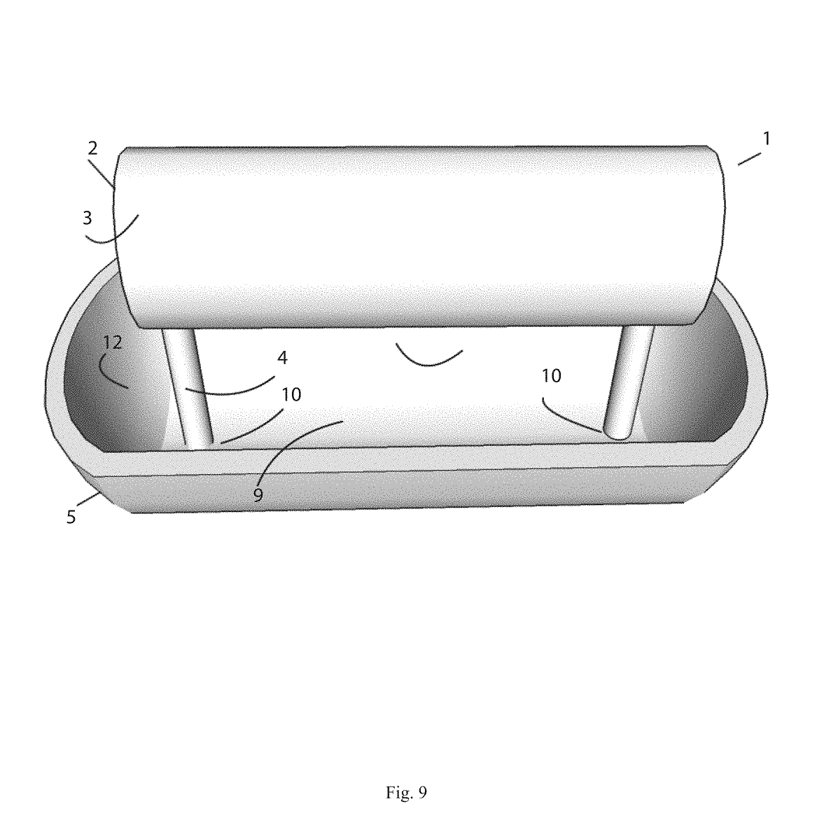

FIG. 10 is a perspective view which shows the handle and connector arms.

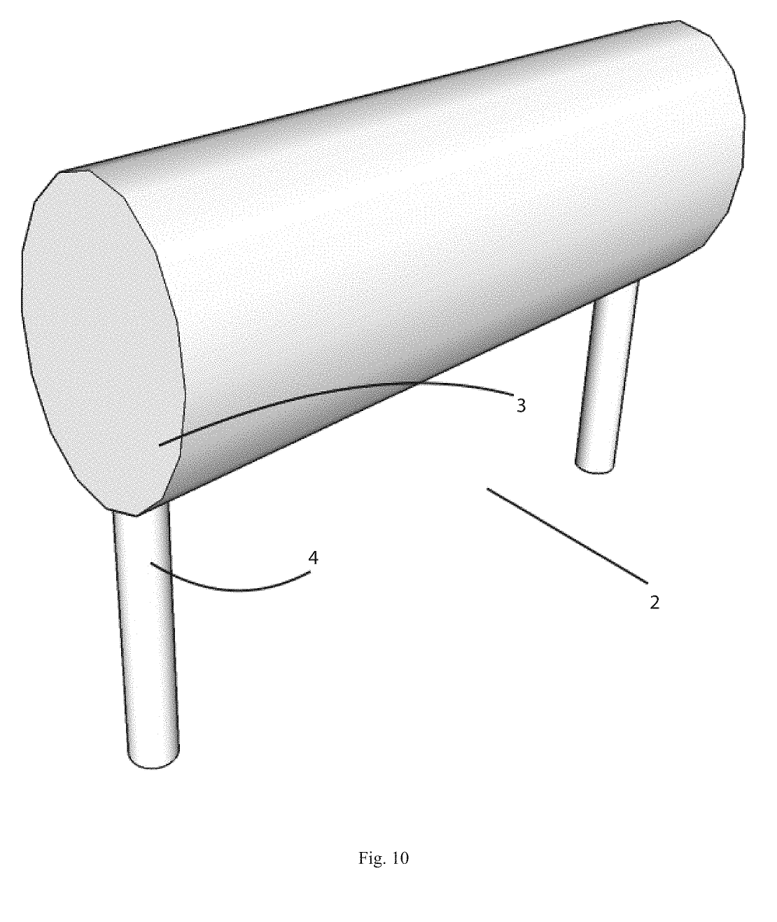

FIG. 11 is a perspective view which shows the pressure block without handles.

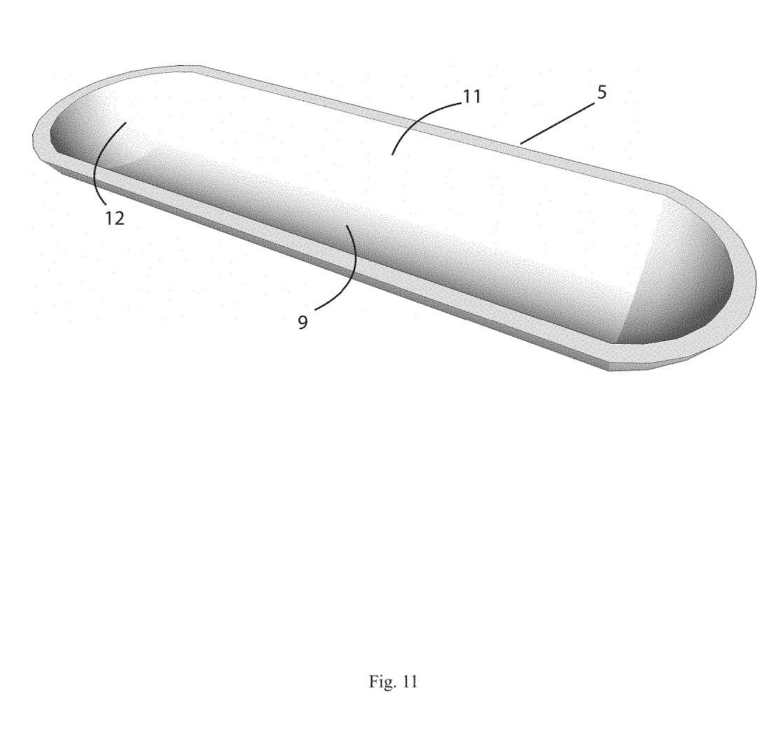

FIG. 12 is a perspective view which shows the pressure block with a long handle.

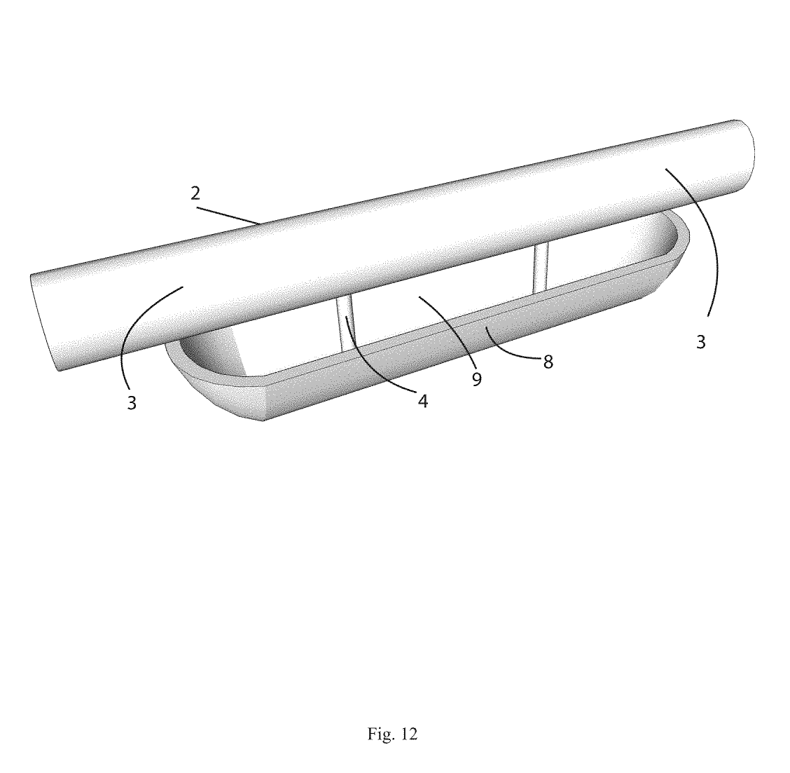

FIG. 13 is a perspective view which shows the pressure block with two perpendicular handles.

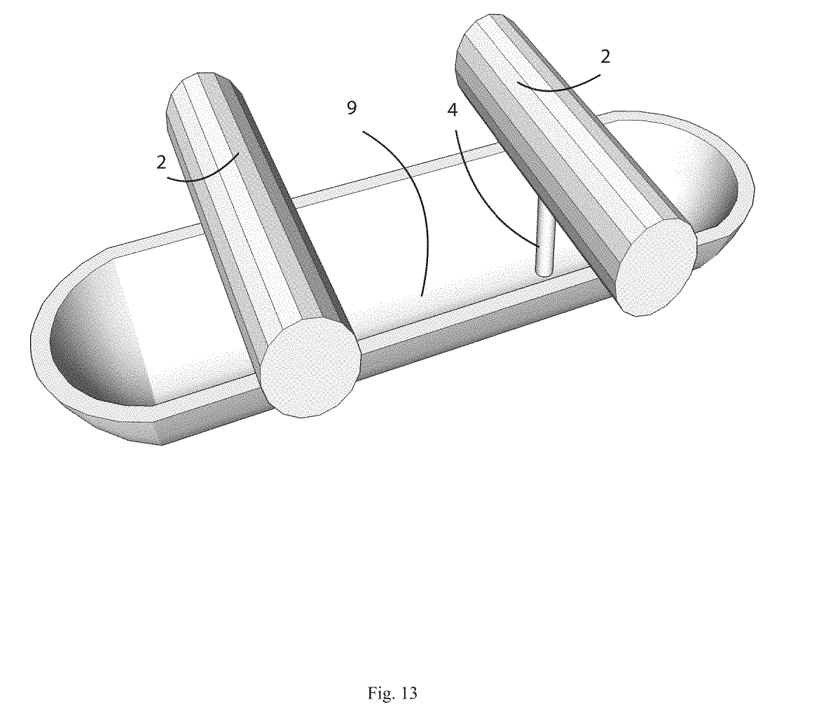

FIG. 14 is a perspective view which shows the pressure block with one perpendicular handle.

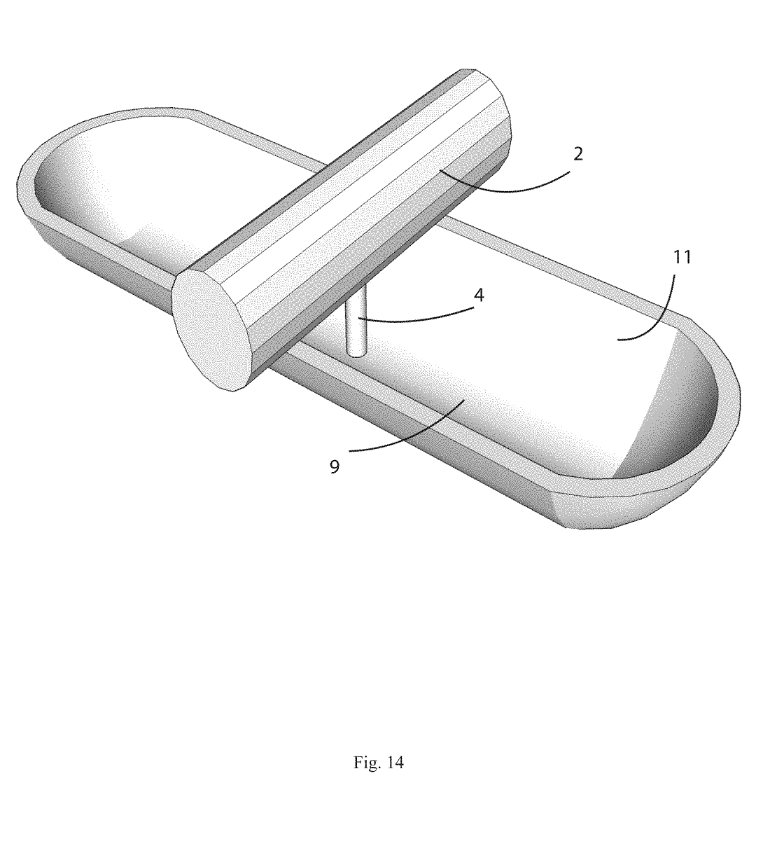

FIG. 15 is a perspective view which shows the pressure block with a convex dorsal surface.

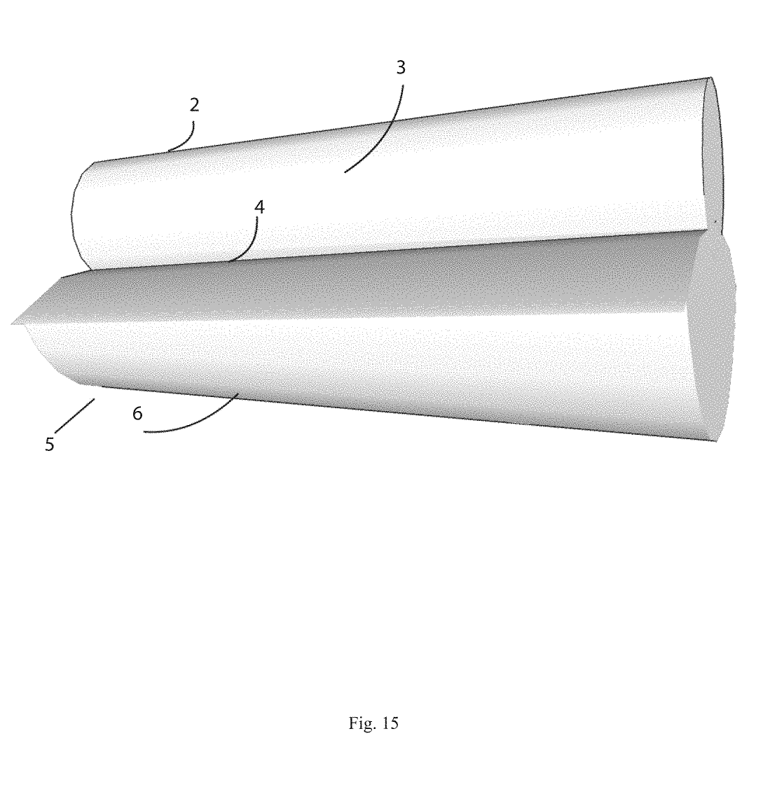

### DETAILED DESCRIPTION OF THE INVENTION

A preferred embodiment of the present invention is now described with reference to the figures, where like reference numbers indicate identical or functionally similar elements. Also in the figures, the leftmost digit of each reference number corresponds to the figure in which the reference number is first used. While specific configurations and arrangements are discussed, it should be understood that this is done for illustrative purposes only. A person of ordinary skill in the relevant art will recognize that other configurations and arrangements can be used without departing from the spirit and scope of the invention. It will be apparent to a person of ordinary skill in the relevant art that this invention can also be employed in a variety of other systems and applications.

The invention has some elements that are commonly known and terms that are defined for tech specification including: a pressure designated region 13, a pressure augmented scoping procedure 14, an organ 15, a patient 16, and a scope 17. However their use and relationships to the novel components and steps of the invention render them applicable herein. In order to preface the roles they play in the specification, they are subsequently explained here.

In general, the invention is used as follows: First, a team of medical professionals is required to do a pressure augmented scoping procedure (Step 1). The term pressure augmented scoping procedure 14 comprises a medical procedure in which a scope is guided through one or more organs. This procedure also has the characteristic that pressure is usually applied from outside the body in order to aid scope navigation. In some embodiments, it is thought that an example of a pressure augmented scoping procedure 14 could be a colonoscopy or perhaps a cystoscopy and the like.

Then, one or more of said medical professional obtains a pressure paddle 1 the instance invention, herein. The pressure paddle 1 preferably comprises a couple subelements; a handle 2 and a pressure block 5. Next, one or more medical professionals performs a pressure augmented scoping procedure when a first medical professional enters an organ 15 with the scope 17.

An organ 15 for the purposes of this specification, comprises an organ that is the target of a medical procedure which uses a scope aided by outside pressure. In some embodiments, it is thought that an example of an organ 15 could be a colon or perhaps a bladder and the like. The scope 17 comprises a long, thin, flexible tube with a camera and a light on the end used for medical procedures by threading through one or more colons or bladders.

While attempting to guide the scope 17 through the organ 15, a first medical professional indicates that pressure should be applied to the organ at a pressure designated region 13 to help guide the scope to one or more second medical professional. The pressure designated region 13 comprises a region that if pushed down upon will collapse an inflated organ, such as a colon and may include any intervening clothes or linens worn by the patient. In some embodiments, it is thought that an example of a pressure designated region 13 could be skin, skin though clothes, or sheets clothes and the like.

Next, a second medical professional grasps one or more pressure paddle 1 on the handle 2. The handle 2 comprises a part of, or attachment to, an object that can be moved or used by hand that is connected to the superior surface 9 of the pressure paddle. In some embodiments, it is thought that if the handle 2 is absent then one may push down on the back of the superior surface 9 with the palm of their hand. The handle 2 preferably comprises the grip 3 and the connector arms 4. The handle 2 has multiple alternative embodiments herein termed the ‘altered orientation’ embodiment, the ‘no handle’ embodiment, the ‘two handles’ embodiment, and the ‘long handle two hands’ embodiment. The ‘no handle’ embodiment is one where the handle is not present, only the pressure block 5. The ‘two handles’ embodiment is one where two or more handles are perpendicular or at a different angle than parallell with the pressure block 5. The ‘long handle two hands’ embodiment is an embodiment where a long handle allows one to use two hands with the pressure block 5.

Spatially, the grip 3 is preferably positioned within the handle 2 and above the superior surface 9. The grip 3 is mainly thought to be composed of plastic as this allows the grip 3 to be easily cleaned. However other embodiments may be composed of any of the following: rubber, foam, metal, or composites. The grip 3 is preferably shaped like cylinder however, it is thought that in alternative embodiments that it may also be shaped like a flat rectangle. In some embodiments, the grip 3 has a preferred length of 5 inches but in other embodiments, may range from a minimum of 2 inches to a maximum length of 12 inches. In some embodiments, the grip 3 has a preferred diameter of 0.75 inches but in other embodiments, may range from a minimum of 0.25 inches to a maximum diameter of 3 inches. The grip 3 comprises the part of the device where a person grasps and wraps there fingers around it in order to apply pressure and relocate the position of the device. In some embodiments, it is thought that if the grip 3 is absent then one may push down on the back of the superior surface 9 with the palm of their hand. The connector arms 4 comprises the arms of the handle that operably connect the grip 3 to the superior surface 9. In some embodiments, it is thought that if the connector arms 4 are absent then one may use the device without a handle 2 or grip 3 or connector arms 4.

When grasping the pressure paddle 1, the second medical professional's hand lies adjacent to the superior surface 9 of the pressure block 5. Like the handle 2, the pressure block 5 is mainly thought to be composed of plastic, however other embodiments may be composed of any of the following: rubber, foam, metal, or composites. The pressure block 5 is preferably shaped like a rectangle with curved edges, however, it is thought that in alternative embodiments that it may also be shaped like a circle, an oval, a rectangle, or a square. In some embodiments, the pressure block 5 has a preferred length of 6 inches but in other embodiments, may range from a minimum of 1.5 inches to a maximum length of 12 inches. In general, the preferred length value can be calculated by estimating the amount of length that is an inch or more larger than an adult hand. In general, the maximum length value can be calculated by estimating the amount of length that allows pressure to be applied on the colon without impacting other structures, such as the ribcage or pelvis. In general, the minimum length value can be calculated by estimating the amount of length that still allows pressure to direct the colonoscope in the desired vector.

In some embodiments, the pressure block 5 has a preferred thickness of 0.75 inches but in other embodiments, may range from a minimum of 0.1 inches to a maximum thickness of 6 inches. In some embodiments, the pressure block 5 has a preferred diameter of 5 inches but in other embodiments, may range from a minimum of 1 inches to a maximum diameter of 7.5 inches. In general, the preferred value can be calculated by estimating the amount the average width of the patient colon, which may vary dependent on age, size, pathology etc. The pressure block 5 comprises a curved block that allows one to have a focused enough curve in order to progress through intervening tissue more easily and apply pressure to colon. The pressure block 5 preferably comprises the contact surface 6 and the superior surface 9. The pressure block 5 has an alternative embodiment herein termed the ‘filled recess’ embodiment. The ‘filled recess’ embodiment is an embodiment where the superior surface 9 is not concave, but convex.

The superior surface 9 comprises the top surface of the pressure block 5. It is preferably convex-shaped however, it is thought that in alternative embodiments that it may also be shaped like a football. The superior surface 9 preferably comprises connector arm binding regions, a dorsal lateral curved surface 11, and finally dorsal anter/posterior curved surfaces. The connector arm binding regions 10 comprises the regions of the superior surface 9 where connector arms 4 bind to the pressure block 5.

The dorsal lateral curved surface 11 comprises the curved surfaces along the superior surface that run parallel with the pressure block 5. In some embodiments, it is thought that an example of a dorsal lateral curved surface 11 may include an angled surface with a vertex and the like. The dorsal anter/posterior curved surfaces 12 comprises the curved surfaces along the superior surface that run perpendicular with the pressure block 5. In some embodiments, it is thought that an example of dorsal anter/posterior curved surfaces may include an angled surface with a vertex and the like.

After grasping the pressure paddle 1 by the handle 2, a second medical professional orients the pressure paddle 1 in the same longitudinal direction as the organ at the pressure designated region 13. Then the second medical professional places the contact surface 6 on to the pressure designated region 13 of the patient and applies downward pressure.

The contact surface 6 comprises the bottom surface of the pressure block 5 which contacts the pressure designated area. The contact surface 6 preferably comprises the ventrolateral curved surface 7 and the ventral anter/posterior curved surfaces 8. The ventrolateral curved surface 7 comprises the curved surfaces along the contact surface that run in parallel with the pressure block 5. In some embodiments, it is thought that if the ventrolateral curved surface 7 is absent then may be an angled surface with a vertex with the contact surface. The ventral anter/posterior curved surfaces 8 comprises the curved surfaces along the contact surface that run perpendicular with the pressure block 5. In some embodiments, it is thought that if the ventral anter/posterior curved surfaces 8 is absent then may be an angled surface with a vertex with the contact surface.

I claim:

1. A device for a medical procedure comprising;
a pressure block, said pressure block having a contact surface and a superior surface, and said pressure block having a curve, where in said curve diameter is between 0.8 and 1.5 times greater than the target patient colon cross sectional width.

2. The device of claim 1 wherein said pressure block is operably attached to a handle, said handle having a grip and connector arms;

3. The device of claim 2 wherein said pressure block has a length that is between 1 and 1.2 times greater than the palm width of the medical professional who is using it.

4. The device of claim 3 wherein said pressure block has a radius having a curve wherein said curve has a radius between 0.5 and 3.75 inches.

5. The device of claim 4 wherein said pressure block has a length between 1.5 and 12 inches.

6. The device of claim 5 wherein said pressure block has a thickness between 0.1 and 6 inches.

7. The device of claim 6 wherein said pressure block is made from plastic, rubber, foam, metal, or composites.

8. The device of claim 7 wherein said pressure block is made from plastic.

9. The device of claim 2 wherein said handle is longer than the pressure block

10. The device of claim 1 comprising one or more handles oriented perpendicular to the pressure block
